(19) United States
(12) Patent Application Publication
(10) Pub. No.: US 2026/0337655 A1
(43) Pub. Date: Oct. 29, 2026

(54) LEUCINE RECODING SCHEME

(71) Applicant: Constructive Biology Limited, Whittlesford (GB)

(72) Inventors: Jamie Choy Ling LUM, Whittlesford (GB); Dionysis GRIGORIADIS, Whittlesford (GB); Daniel DE LA TORRE, Whittlesford (GB)

(21) Appl. No.: 19/181,650

(22) Filed: Apr. 17, 2025

(51) Int. Cl. C12P 21/02; C12N 15/70

(52) U.S. Cl. CPC C12P 21/02; C12N 15/70; C12N 2800/101

(57) ABSTRACT

The present invention relates to a DNA sequence encoding one or more essential genes of an E. coli organism, said DNA sequence comprising at least 17 kbp and being recoded such that a leucine codon is changed to an alternative codon, wherein said leucine codon is CTT or CTC; and to E. coli comprising such a sequence, including E. coli which has been recoded to replace all instances of CTT and/or CTC.

### FIELD OF THE INVENTION

The present disclosure relates to recoding schemes for genome engineering, and more particularly to recoding schemes suitable for recoding the amino acid leucine genome-wide.

### BACKGROUND TO THE INVENTION

The design and synthesis of genomes provides a powerful approach for understanding and engineering biology. Genome synthesis has the potential to accelerate metabolic engineering. In particular, genome synthesis has the potential to elucidate synonymous codon function and to facilitate genetically encoded unnatural polymer synthesis (Wang, K., et al., 2016. Nature, 539 (7627), 59-64).

The standard genetic code encodes the 20 canonical amino acids using 61 sense codons, and eighteen of the twenty amino acids are encoded by more than one synonymous codon. Nature chooses one sense codon, from up to six synonyms, to encode each amino acid at each position in a gene. Synonymous codon choice can influence mRNA folding, transcriptional and translational regulatory sequences, translation rate, co-translational folding, protein levels, and has emerging and yet to be understood roles (Wang, K., et al., 2016. Nature, 539 (7627), 59-64; and Cambray, G., et al., 2018. Nature biotechnology, 36 (10), 1005-1015).

Genome-wide replacement of a target codon with synonymous codons (synonymous codon compression) may provide a foundation for reassigning sense codons to non-canonical amino acids (or other monomers) to facilitate the in vivo biosynthesis of genetically encoded non-canonical biopolymers (Chin, J. W., 2017. Nature, 550 (7674), 53-60).

Site-directed mutagenesis approaches have been used to replace up to 321 amber stop codons in the E. coli genome (Mukai, T., et al., 2015. Scientific reports, 5, p. 9699). However, sense codons are commonly orders of magnitude more abundant than stop codons, and genome synthesis, rather than mutagenesis, may be the preferred route to tackling sense codon removal in many cases.

Genome synthesis has enabled the creation of Mycoplasma with synthetic genomes (Gibson, D. G., et al., 2010. Science, 329 (5987), 52-56) and the creation of nine strains of S. cerevisiae in which the DNA for one or two of the sixteen chromosomes is replaced by synthetic DNA (Zhang, W., et al., 2017. Science, 355 (6329), eaaf3981; and Richardson, S. M., et al., 2017. Science, 355 (6329), 1040-1044). These experiments have replaced up to 1 Mb of DNA (0.99 Mb, yeast; 1.08 Mb, Mycoplasma) in individual strains. Replicon excision for enhanced genome engineering through programmed recombination (REXER) has been reported for replacing>100 kb of the E. coli genome with synthetic DNA in a single step. Moreover, it has been shown that REXER can be iterated via genome stepwise interchange synthesis (GENESIS) to replace 220 kb of the E. coli genome with 230 kb of synthetic DNA (Wang, K., et al., 2016. Nature, 539 (7627), 59-64; WO 2018/020248).

Genome synthesis has been used to alter synonymous codons in individual genes (Napolitano, M. G., et al., 2016. PNAS, 113 (38), E5588-E5597), genomic regions and essential operons (Wang, K., et al., 2016. Nature, 539 (7627), 59-64; and Lau, Y. H., et al. 2017. Nucleic acids research, 45 (11), 6971-6980). For instance, Wang et al. used defined ‘recoding schemes’ to replace a ~20 kb region of the E. coli genome rich in both essential genes and target codons.

However, recoding of leucine codons has not been successfully carried out in E. coli. Wang et al., for instance, were unable to recode leucine, with none of the attempted recoding schemes being successful. Lau et al. did apparently successfully recode leucine, but in Salmonella typhimurium; the recoding scheme used by Lau et al. was not successful in E. coli, as attempted by Wang or by Ostrov (Ostrov N, et al., Science. 2016; 353 (6301): 819-822). UUA and UUG leucine codons were recoded in an attempt to create a 57-codon E. coli, but the authors were unable to reassemble a single genome (Nyerges et al, BioRxiv 2024 https://doi.org/10.1101/2024.06.16.599206).

Wang, Ostrov and Nyerges attempted to recode the codons UUA and UUG. These codons are straightforward to replace, requiring only single base changes to alter to for example CUA and CUG. However, in Nyerges, strains containing partial replacements a subset of UUA and UUG codons in a ~350 kbp region of the genome (Segments 1-8) results in a >2.5-fold increase in doubling time with respect to the non-recoded parent, and neither of these codons can be successfully recoded in a complete E. coli genome. Moreover, organisms comprising the recoded DNA ins segments 1-8 display inhibited growth (see Supplementary Table 3 of Nyerges et al, 2024).

E. coli strains in which all genes have been substantially recoded have now been described. Fredens, J., et al., Nature 569, 514-518 (2019) and Robertson et al. Science. 2021 Jun. 4; 372 (6546): 1057-1062 describe recoded E. coli in which both sense codons and stop codons have been entirely reassigned. However, neither of these publications describes the successful recoding of leucine codons.

We define herein a successful recoding scheme for leucine in E. coli, opening the possibility of recoding of leucine in a recoded E. coli strain.

### SUMMARY OF THE INVENTION

According to a first aspect, there is provided a DNA sequence encoding one or more essential genes of an E. coli organism, said DNA sequence comprising at least 17 kbp and being recoded such that a leucine codon is changed to an alternative codon, wherein said leucine codon is CTT or CTC.

The recoding of CTT or CTC has not previously been attempted. Prior attempts have concentrated on the codons TTA or TTG but these efforts have been unsuccessful. We show herein that both CTT and CTC can successfully be recoded.

The alternative codon is preferably a synonymous codon, that is a codon which encodes leucine. Advantageously, CTT is recoded to TTG or preferably TTA. CTC is preferably recoded to TTG.

Preferably, both CTT and CTC are recoded. A preferred recoding scheme is CTT-TTA and CTC-TTG. CTT-TTG and CTC-TTG is also possible.

In one embodiment, the DNA sequence of the invention encodes at least 10 essential genes. The DNA sequence is at least 17 kbp in length, and may be 20, 30, 40, 50, 60, 70, 80, 90 or 100 Kb in length. Longer sequences can be recoded; the insert size of the BAC places a practical limit of about 300 kb on the potential length.

The DNA sequence can be used to replace a homologous region of the E. coli genome. Thus, the invention provides an engineered E. coli organism wherein at least one section of the endogenous genome has been replaced with a DNA sequence according to the invention. The DNA sequence and the replaced E. coli genome section are advantageously the same length.

In some embodiments, two or more sections of the endogenous genome have been replaced with a DNA sequence according to the invention. Two or more sections are replaced when two replacement events are needed to replace the sections; the sections may be contiguous, or separate in the genome.

A recoded codon, which has been replaced with a synonymous codon, can be reassigned to a non-canonical monomer such as a non-canonical amino acid.

Thus, an engineered E. coli organism can be provided, comprising a recoded genome wherein a leucine codon at all instances within a template genome is changed to an alternative codon and said leucine codon is reassigned to a non-canonical amino acid, wherein said leucine codon is CTT or CTC.

In embodiments, an engineered E. coli organism comprising at least one segment of recoded DNA of at least 17 kbp in length has growth characteristics which are substantially identical to the equivalent wild-type E. coli strain without the recoded DNA.

As used herein, “all instances” includes replacement of substantially all instances, whereby some codons may remain unrecoded. Advantageously, said codons are in non-essential genes.

Accordingly, in one aspect the present invention provides an engineered E. coli genome comprising 5 or fewer occurrences of one or more leucine codons selected from CTT and CTC. In some embodiments the synthetic prokaryotic genome comprises 4 or fewer, 3 or fewer, 2 or fewer, 1 or fewer, or no occurrences of said leucine codons. In some embodiments the one or more leucine codons consist of one leucine codon or two leucine codons, preferably two leucine codons. In some embodiments the synthetic prokaryotic genome comprises no occurrences of said two leucine codons, and no occurrences of one stop codon, preferably the amber stop codon (TAG).

In embodiments, the engineered E. coli genome of the invention is comprised in an engineered E. coli organism. The engineered E. coli of the invention can be sued to expresses proteins comprising non-canonical amino acids. The engineered organism of the invention, in some embodiments, does not comprise the cognate tRNA to said leucine codon which is changed to an alternative codon in the recoded genome. Preferably, both leucine codons CTT and CTC are removed and the LeuU tRNA gene is not present in the recoded genome.

In embodiments, leuW (UAG) can also be removed; in this case, however, leuP (CAG) is engineered to also read CTA. Alternatively, leuW itself is engineered (rather than removed) to not read CTT and CTC

In embodiments, the alternative codon is a synonymous codon. TTA and TTG are preferred, as set forth above.

In embodiments, further sense codons may be recoded. For example, all instances of one or more serine codons may be recoded, including in particular TCA and TCG may be recoded.

In embodiments, all instances of at least one stop codon are removed and the cognate release factor is removed. Preferably, the stop codon it TAG and the release factor is eRF1.

The nucleic acids and organisms according to the invention are useful for the production of polypeptides comprising one or more non-canonical monomers, such as non-canonical amino acids. Exemplary non-canonical monomers include non-canonical amino acids, non-alpha-amino acids which are monomers that lack the RCH (NH2) COOH structure of an amino acid, and in particular lack the alpha (NH2) group. Alpha hydroxy acids are exemplary non-alpha-amino acids.

The methods and organisms of the invention may be used to produce polypeptides comprising one, two, three, four or five non-canonical amino acids, said non-canonical amino acids being different. More than one of each different non-canonical amino acid may be included, such that a polypeptide may comprise 10, 20, 30, 40, 50 or more non-canonical amino acids.

### BRIEF DESCRIPTION OF THE FIGURES

FIGS. 1A and 1B show growth curves of E. coli MDS42 which comprise the recoded E. coli MDS42 cell division operon (from 89,062 to 106,507). FIG. 1A illustrates the growth of clone CDO_Leu_9 and FIG. 1B illustrates the growth curve of CDO_Leu_12, which incorporate CTT-TTG and CTC-TTG (Leu_9) and CTT-TTA and CTC-TTG Leu_12) mutations respectively. The Curves of the mutant clones is shown in the daker shade; the light shade is MDS42. The growth characteristics of mutant and wild-type organisms are substantially identical.

### DETAILED DESCRIPTION OF THE INVENTION

Methods for recoding genomes of organisms generally involve iterative replacement of sections of the genome with corresponding sections recoded nucleic acid. In that way, multiple organisms can be produced in which a part of the genome has been recoded. Alternatively, nucleic acid can be iteratively added to episomal replicons and then assembled into genomes. For such approaches, the recoding of large sections of DNA is required to provide the genome sections form which a recoded genome can be constructed.

### Recoded Polynucleotides

The present invention provides polynucleotides comprising one or more essential genes, said polynucleotides being recoded such that one or more leucine codons has been replaced with a synonymous codon.

Leucine has six codons, comprising UUA, UUG, CUA, CUG, CUU, and CUC; in the genome, these are encoded in DNA as TTA, TTG, CTA, CTG, CTT and CTC.

In the present invention, the codons CTT and/or CTC are replaced. We have found that synonymous replacement of these codons, preferably with TTG or TTA, leads to successful recoding of DNA and viable recoded organisms.

The present inventors identified the E. coli cell division operon (see Wang et al (2016)) as an ideal target to test these synonymous recoding schemes because it i) is rich in essential genes (12 out of 15 genes in the region are essential), ii) contains genes expressed at a range of levels, iii) includes membrane proteins (a class of proteins for which co-translational folding is known to be affected by synonymous codon choice), iv) includes several proteins that interact and for which the ratios of proteins expressed are distinct and crucial (which will favour intergenic epistatic interactions amongst codons), and v) is rich in the target leucine codons. Wang et al (2016) anticipated that these features would ensure that the region captures important properties of the genome, and that the success and failure of synonymous recoding in the region, would be reflected in viability and growth.

As demonstrated by Fredens et al (2019), recoding schemes which are successful in recoding a segment comprising essential genes such as the cell division operon can be applied to the entire E. coli genome. Accordingly, the recoding scheme of the present invention can be applied to any region of the E. coli genome, or indeed the entire genome.

The polynucleotide or DNA sequence according to the invention which is recoded to remove leucine codons CTT and/or CTC is at least 17 kbp in length. In some embodiments, smaller sections of DNA may be recoded and assembled into sections of 17 kbp or more; preferably, however, longer sections of DNA of at least 17 kbp are recoded and themselves assembled into longer DNA sections up to entire genomes.

Preferably, the DNA sequence is 15 kb, 20 kb, 30 Kb, 50 kb, 75 kb, 100 kb, 200 kb or up to 300 kb in length. Recoded DNA can be encoded in a BAC or YAC in insert lengths of up to 300 kb, though the capacity of a YAC insert is theoretically higher (up to 2000 kb).

The polynucleotide comprises one or more E. coli essential genes. A gene is “essential”, as used herein, if the product of the gene is required for viability of the E. coli cell. For instance, if the prevention of expression of a functional form of a protein encoded by a gene would result in non-viability of the cell, then the gene is considered essential. In particular, a gene is considered essential if a “blank” codon (i.e. a codon for which the cell contains no corresponding tRNA or release factor) within the gene results in a loss of cell viability.

Therefore, in an embodiment, all of the genes of the prokaryotic cell for which a blank codon could not be tolerated without a loss of viability are recoded, but genes that are able to tolerate blank codons may not be recoded. Thus, the skilled person can assess whether all of the essential genes have been recoded by assessing whether a cognate tRNA is dispensable.

In particular embodiments, the DNA sequence comprises 10, 20, 30 or more essential genes with no occurrences of the first and/or second type of leucine codon. The first and second types of leucine codons are CTT and CTC respectively. In embodiments, the engineered E. coli organism comprises 100 or more, 200 or more, or 300 or more essential genes with no occurrences of the first and/or second type of leucine codon. For instance, all or substantially all of the essential genes in the genome of the engineered E. coli may comprise no occurrences of the first and/or second type of leucine codon.

In some embodiments, the essential genes may be selected from one or more of the list consisting of: ribF, IspA, ispH, dapB, folA, imp, yabQ, ftsL, ftsl, murE, murF, mraY, murD, ftsW, murG, murC, ftsQ, ftsA, ftsZ, IpxC, secM, secA, can, folk, hemL, yadR, dapD, map, rpsB, tsf, pyrH, frr, dxr, ispU, cdsA, yael, yaeT, IpxD, fabZ, IpxA, IpxB, dnaE, accA, tilS, proS, yafF, hemB, secD, secF, ribD, ribE, thiL, dxs, ispA, dnaX, adk, hemH, IpxH, cysS, folD, entD, mrdB, mrdA, nadD, holA, ripB, leuS, Int, ginS, fldA, cydA, infA, cydC, ftsK, lolA, serS, rpsA, msbA, IpxK, kdsB, mukF, mukE, mukB, asnS, fabA, mviN, rne, fabD, fabG, acpP, tmk, holB, loIC, loID, loIE, purB, minE, minD, pth, prsA, ispE, lolB, hemA, prfA, prmC, kdsA, topA, ribA, fabl, tyrS, ribC, ydiL, pheT, pheS, rpIT, infC, thrS, nadE, gapA, yeaZ, aspS, argS, pgsA, yefM, metG, folE, yejM, gyrA, nrdA, nrdB, folC, accD, fabB, gltX, ligA, zipA, dapE, dapA, der, hisS, ispG, suhB, tadA, acpS, era, rnc, lepB, rpoE, pssA, yfiO, rpIS, trmD, rpsP, ffh, grpE, csrA, ispF, ispD, ftsB, eno, pyrG, chpR, Igt, fbaA, pgk, yqgD, metK, yqgF, plsC, ygiT, parE, ribB, cca, ygjD, tdcF, yraL, yhbV, infB, nusA, ftsH, obgE, rpmA, rpIU, ispB, murA, yrbB, yrbK, yhbN, rpsi, rpIM, degS, mreD, mreC, mreB, accB, accC, yrdC, def, fmt, rplQ, rpoA, rpsD, rpsK, rpsM, secY, rplO, rpmD, rpsE, rplR, rplF, rpsH, rpsN, rplE, rpiX, rpIN, rpsQ, rpmC, rpIP, rpsC, rpIV, rpsS, rplB, rplW, rpID, rpIC, rpsJ, fusA, rpsG, rpsL, trpS, yrfF, asd, rpoH, ftsX, ftsE, ftsY, yhhQ, bcsB, glyQ, gpsA, rfaK, kdtA, coaD, rpmB, dfp, dut, gmk, spoT, gyrB, dnaN, dnaA, rpmH, rnpA, yidC, tnaB, gimS, glmU, wzyE, hemD, hemC, yigP, ubiB, ubiD, hemG, yihA, ftsN, murl, murB, birA, secE, nusG, rplJ, rpIL, rpoB, rpoC, ubiA, plsB, lexA, dnaB, ssb, alsK, groS, psd, orn, yjeE, rpsR, chpS, ppa, valS, yjgP, yjgQ, and dnaC.

In particular, the essential genes may be selected from one or more of the list consisting of: ribF, IspA, ispH, dapB, folA, imp, yabQ, IpxC, secM, secA, can, folk, hemL, yadR, dapD, map, rpsB, tsf, pyrH, frr, dxr, ispU, cdsA, yaeL, yaeT, IpxD, fabZ, IpxA, IpxB, dnaE, accA, tilS, proS, yafF, hemB, secD, secF, ribD, ribE, thiL, dxs, ispA, dnaX, adk, hemH, IpxH, cysS, folD, entD, mrdB, mrdA, nadD, holA, ripB, leuS, Int, ginS, fldA, cydA, infA, cydC, ftsK, lolA, serS, rpsA, msbA, IpxK, kdsB, mukF, mukE, mukB, asnS, fabA, mviN, rne, fabD, fabG, acpP, tmk, holB, loIC, loID, loIE, purB, minE, minD, pth, prsA, ispE, lolB, hemA, prfA, prmC, kdsA, topA, ribA, fabl, tyrS, ribC, ydiL, pheT, pheS, rpIT, infC, thrS, nadE, gapA, yeaZ, aspS, argS, pgsA, yefM, metG, folE, yejM, gyrA, nrdA, nrdB, folC, accD, fabB, gltX, ligA, zipA, dapE, dapA, der, hisS, ispG, suhB, tadA, acpS, era, rnc, lepB, rpoE, pssA, yfiO, rpIS, trmD, rpsP, ffh, grpE, csrA, ispF, ispD, ftsB, eno, pyrG, chpR, Igt, fbaA, pgk, yqgD, metK, yqgF, plsC, ygiT, parE, ribB, cca, ygjD, tdcF, yraL, yhbV, infB, nusA, ftsH, obgE, rpmA, rpIU, ispB, murA, yrbB, yrbK, yhbN, rpsl, rpIM, degS, mreD, mreC, mreB, accB, accC, yrdC, def, fmt, rplQ, rpoA, rpsD, rpsK, rpsM, secY, rplO, rpmD, rpsE, rpIR, rpIF, rpsH, rpsN, rpIE, rpIX, rpIN, rpsQ, rpmC, rpIP, rpsC, rpIV, rpsS, rplB, rplW, rpID, rpIC, rpsJ, fusA, rpsG, rpsL, trpS, yrfF, asd, rpoH, ftsX, ftsE, ftsY, yhhQ, bcsB, glyQ, gpsA, rfak, kdtA, coaD, rpmB, dfp, dut, gmk, spoT, gyrB, dnaN, dnaA, rpmH, rnpA, yidC, tnaB, gimS, glmU, wzyE, hemD, hemC, yigP, ubiB, ubiD, hemG, yihA, ftsN, murl, murB, birA, secE, nusG, rplJ, rpIL, rpoB, rpoC, ubiA, plsB, lexA, dnaB, ssb, alsK, groS, psd, orn, yjeE, rpsR, chpS, ppa, valS, yjgP, yjgQ, and dnaC.

Suitable E. coli cells for use in the present invention include K-12, MG1655, BL21, BL21 (DE3), AD494, Origami, HMS174, BLR (DE3), HMS174 (DE3), Tuner (DE3), Origami2 (DE3), Rosetta2 (DE3), Lemo21 (DE3), NiCo21 (DE3), T7 Express, SHuffle Express, C41 (DE3), C43 (DE3), and m15 pREP4 or derivatives thereof (Rosano, G. L. and Ceccarelli, E. A., 2014. Frontiers in microbiology, 5, p. 172). In particular, the cell may be MG1655 or BL21, or a derivative thereof. MG1655 is considered as the wild type strain of E coli. The GenBank ID of genomic sequence of this strain is U00096. BL21 is widely available commercially. For example, it can be purchased from New England BioLabs with catalog number C2530H.

Any such E. coli cell can be used as a parental strain from which a recoded engineered E. coli according to the invention can be prepared.

A “parental strain” is a strain that existed or was created as part of the development of the E. coli cell of the invention. Hence, the E. coli cell of the invention is derived, directly or indirectly, from the parental strain. A parental strain may also be referred to as a “progenitor strain”.

The parental strain can also be MDS42 or Syn61 (GenBank accession number CP040347.1), including derivatives of Syn61 such as Syn61A3 (see WO2022/248061).

The methods of inducing mutagenesis, maintaining the cells, and selecting the cell may be any as disclosed herein or in WO2018/020248, WO2020/229592, WO2022/248061 or WO2023/078997.

### Methods for Recoding a Genome

The following methods enable the skilled person to recode genomes to replace sense or stop codons with synonymous codons. Further information is available in: i) K. Wang et al., Defining synonymous codon compression schemes by genome recoding. Nature 539, 59-64 (2016); ii) J. Fredens et al., Total synthesis of Escherichia coli with a recoded genome. Nature 569, 514-518 (2019); iii) WO2018/020248, and iv) WO2020/229592 (each of which is incorporated herein by reference).

In an example, preferably one or more rounds of recombination-mediated genetic engineering are used to edit 10-1000 kb, 50-1000 kb, 100-1000 kb, or 100-500 kb of a parent genome to provide two or more different partially synthetic genomes. Thus, in preferred examples each round of recombination-mediated genetic engineering inserts or replaces 10 kb or more, 50 kb or more, 100 kb or more, or about 100 kb of DNA in the parent genome.

As used herein, the term “recombination-mediated genetic engineering” (also known as “recombineering”) is a method for genetic engineering (i.e. editing genomes) based on homologous recombination systems. Typically recombineering is based on homologous recombination in Escherichia coli mediated by bacteriophage proteins, either RecE/RecT from Rac prophage or Redαβδ from bacteriophage lambda. Any suitable method of recombination-mediated genetic engineering may be used. Methods for recombination-mediated genetic engineering will be well known to those of skill in the art.

In “classical recombination” (exemplified by lambda red mediated recombination in E. coli), short regions of synthetic DNA may be inserted into the genome or used to replace genomic DNA in a two-step process: i) transformation of cells with linear double stranded DNA (dsDNA) carrying a stretch of synthetic DNA, coupled with a positive selection marker, and flanked by a homology region (HR) to the target region of the genome on each end, and ii) recombination mediated by the homologous regions, followed by selection for genomic integration by virtue of the positive selection marker. This approach can be used to insert or replace 2-3 kb of genomic DNA. Thus, if classical recombination is used, many rounds of recombination-mediated genetic engineering would be required to edit 100-500 kb of the parent genome.

Thus, in preferred examples the one or more rounds of recombination-mediated genetic engineering comprise one or more rounds of replicon excision for enhanced genome engineering through programmed recombination (REXER).

REXER is described in WO 2018/020248. Each round of REXER may be used to insert or replace about 50 kb to 250 kb, or about 100 kb of DNA in the parent genome.

Thus, the one or more rounds of recombination-mediated genetic engineering may comprise:
i) providing a host cell (e.g. E. coli), wherein the host cell comprises an episomal replicon (e.g. a plasmid or a bacterial artificial chromosome) and a target nucleic acid (e.g. the genome), wherein the episomal replicon comprises a donor nucleic acid sequence (i.e. a synthetic region), wherein the donor nucleic acid sequence comprises in order: 5′-homologous recombination sequence 1-sequence of interest-homologous recombination sequence 2-3′, wherein the sequence of interest comprises a positive selectable marker, and wherein the target nucleic acid comprises in order: 5′-homologous recombination sequence 1-negative selectable marker-homologous recombination sequence 2-3 ′;
ii) providing helper protein(s) capable of supporting nucleic acid recombination in said host cell (e.g. lambda Red proteins);
iii) providing helper protein(s) and/or RNAs capable of supporting nucleic acid excision in said host cell (e.g. CRISPR/Cas9 proteins/RNAs);
iv) inducing excision of said donor nucleic acid sequence;
v) incubating to allow recombination between the excised donor nucleic acid and said target nucleic acid; and
vi) selecting for recombinants having incorporated said donor nucleic acid into said target nucleic acid.

For example, selecting for recombinants having incorporated said donor nucleic acid into said target nucleic acid comprises selection for gain of the positive selectable marker of the donor nucleic acid and loss of the negative selectable marker of the target nucleic acid. In embodiments, selection for gain of the positive selectable marker of the donor nucleic acid and loss of the negative selectable marker of the target nucleic acid is carried out simultaneously. In embodiments, said sequence of interest comprises both a positive selectable marker and a negative selectable marker. The negative selectable marker can be selected from the group consisting of sacB (sucrose sensitivity), rpsL (S12 ribosomal protein streptomycin sensitivity), or phe^{ST251A_A294G} (4-chlorophenylalanine sensitivity). The positive selectable marker can be selected from the group consisting of Cm^{R} (chloramphenicol resistance), Kan^{R} (kanamycin resistance), Hyg^{R} (hygromycin resistance), Gentamycin^{R} (gentamycin resistance), or tetracycline^{R} (tetracycline resistance). Suitably the step of selecting for recombinants comprises sequential selection for said positive and negative markers, or sequential selection for said negative and positive markers. The step of selecting for recombinants can comprise simultaneous selection for said positive and negative markers.

The method as described above can further comprise the step of inducing at least one double stranded break in the target nucleic acid sequence, wherein said double stranded break is between said homologous recombination sequence 1 and said homologous recombination sequence 2. Suitably at least two double stranded breaks are induced in the target nucleic acid sequence, wherein each said double stranded break is between said homologous recombination sequence 1 and said homologous recombination sequence 2.

In embodiments, said excised donor nucleic acid begins with said homologous recombination sequence 1 and ends with said homologous recombination sequence 2.

In embodiments, said episomal replicon comprises a negative selectable marker independent of the donor nucleic acid sequence. Suitably said method comprises the further step of selecting for loss of the episomal replicon by selecting for loss of said negative selectable marker independent of the donor nucleic acid sequence. Suitably said episomal replicon comprises in order: excision cut site 1-donor nucleic acid sequence-excision cut site 2. Suitably said target nucleic acid possesses its own origin of replication capable of functioning within said host cell. Suitably said episomal replicon is a plasmid nucleic acid. Suitably said episomal replicon is a bacterial artificial chromosome (BAC). Suitably said target nucleic acid is the host cell genome.

The episomal replicon (e.g. BAC) may be assembled by homologous recombination, for example in S. cerevisiae, as described in Kouprina, N., et al., 2004. Methods Mol Biol 255, 69-89. The assembly may combine: 7-14 stretches of synthetic DNA, each 6-13 kb in length; a selection construct (comprising a negative selection marker and/or a positive selection marker); and a BAC shuttle vector backbone. The stretches of synthetic DNA may collectively correspond to the donor nucleic acid sequence (i.e. the synthetic region) in the episomal replicon, wherein each stretch comprises 80-200 bp of overlapping DNA sequence with each other, and wherein the overlap regions are free of any recoding targets. The stretches may be supplied in pSC101 or pST vectors flanked by suitable restriction sites (e.g. Bsal, Avrll, Spel, or Xbal). Thus, during assembly the synthetic DNA stretches may be excised by digestion with the corresponding restriction enzymes. Assembly of the episomal replicon may be verified by sequencing.

Suitably the two homology regions may be 30-100 bp, or 40-50 bp, or about 50 bp in length. CRISPR/Cas9 machinery may be used to for excision. In some examples the CRISPR/Cas9 machinery comprises Cas9, tracrRNA and two spacer RNAs, wherein the spacer RNAs target the two homology regions for excision. In preferred embodiments, the spacer RNAs are linear double stranded spacers. In other embodiments, the CRISPR/Cas9 machinery comprises Cas9 and two sgRNAs, wherein the sgRNAs target the two homology regions for excision.

Lambda red recombination machinery may be used for recombination. The lambda red recombination machinery may comprise lambda alpha/beta/gamma.

The method may comprise performing one or more rounds of REXER, i.e. the steps as described above with a first donor nucleic acid sequence, choosing further donor sequence(s) contiguous with said first donor nucleic acid sequence, and repeating said steps with said further donor nucleic acid sequence(s) until the partially synthetic genome has been assembled. This is known as genome stepwise interchange synthesis (GENESIS), described in Wang, K. et al., 2016. Nature 539, 59-64 and is shown schematically in FIG. 4 of WO2020/229592.

In preferred examples the donor sequence(s) correspond to regions of the synthetic genome.

Thus, the donor sequence(s) (i.e. synthetic region) may comprise 20 or fewer occurrences of one or both leucine codons; and/or the donor sequence(s) may comprise 10 or more, 20 or more, or 100 or more genes with no occurrences of one or both leucine codons.

The donor sequence(s) (i.e. synthetic region) may be identical to sequences (i.e. non-synthetic regions) of the parent genome except that they have 50 or fewer, 20 or fewer, 10 or fewer, 5 or fewer, or 0 occurrences of each of one or both leucine codons; and/or comprise less than 10%, 5%, 2%, 1%, 0.5%, 0.1% of the occurrences of each of one or both leucine codons, relative to the corresponding region in the parent genome; and/or comprise 10 or more, 20 or more, or 100 or more genes with no occurrences of one or both leucine codons.

The donor sequence(s) (i.e. synthetic region) may also be refactored relative to the sequences (i.e. non-synthetic regions) of the parent genome. For 3',3′ overlaps (i.e. pairs of genes in opposite orientations) a synthetic insert may be inserted between the genes. For 3′,3′ overlaps the synthetic insert may comprise the overlapping region. For 5′, 3′ overlaps (i.e. pairs of genes in the same orientation) a synthetic insert may be inserted between the genes. For 5′, 3′ overlaps the synthetic insert may comprise: (i) a stop codon; (ii) about 20-200 bp, or 20-100 bp, or 20-50 bp, from upstream of the overlapping region; and (iii) the overlapping region. Preferably, the synthetic insert comprises: (i) a stop codon; (ii) about 20 bp from upstream of the overlapping region; and (iii) the overlapping region. In preferred embodiments the stop codon is in frame with the original start site for the downstream gene. Preferably the stop codon is TAA.

Preferably the donor sequence(s) (i.e. synthetic region) are collectively 50-10000 kb, 100-5000 kb, 100-2000 kb, 100-1000 kb, or 100-500 kb in size. Preferably each donor sequence is 50-300 kb, 100-200 kb, or about 100 kb in size.

Accordingly, the donor sequences may each be about 100 kb in size and identical to corresponding sequences of the parent genome, except they comprise no occurrences of one or more sense codons and all pairs of genes which share an overlapping region comprising the one or more sense codons in the parent genome are refactored, wherein the pairs of genes are those in which sense codon replacements would change the encoded protein sequence of both or either of the pair of genes.

In preferred examples the viability of the genome is tested after each round of recombination-mediated genetic engineering. In some examples the sequence of the genome is verified after each round of recombination-mediated genetic engineering.

In some embodiments the genome is a reduced synthetic genome or a minimal synthetic genome. A “reduced genome” is one in which the size of the parent genome has been reduced by removing non-essential genes and/or non-coding regions. A “minimal genome” is a genome which has been reduced to its minimal size whilst remaining viable e.g. by deletion of all non-essential regions of the genome.

### Refactoring

Genomes contain numerous overlapping open reading frames (ORFs), which can be classified as 3′, 3′ (between ORFs in opposite orientations) or 5′, 3′ (between ORFs in the same orientation). The sense codons to be replaced may be found within both classes of overlap in the parent genome.

If the replacement of the sense codons of each ORF within an overlap can be achieved without changing the encoded protein sequence of either ORF (i.e. by introducing synonymous codon(s)) then it may not be necessary to edit (e.g. refactor) the parent genome. However, when the encoded protein sequence is changed by the replacement of the sense codons, (i.e. one or more synonymous sense codons are not introduced into one or both of the ORFs), then it may be necessary to edit (e.g. refactor) the parent genome.

Thus, in some examples one or more pairs of genes which share an overlapping region comprising the sense codons in the parent genome are refactored. “Refactored” means that the genes are reorganised to prevent changes to the encoded protein sequences. Preferably, the pairs of genes are those in which sense codon replacements (e.g. defined synonymous codon replacements) would change the encoded protein sequence of both or either of the pair of genes. Most preferably, all pairs of genes which share an overlapping region comprising the sense codons in the parent genome are refactored, wherein the pairs of genes are those in which sense codon replacements (e.g. defined synonymous codon replacements) would change the encoded protein sequence of both or either of the pair of genes.

For 3′,3′ overlaps (i.e. pairs of genes in opposite orientations) a synthetic insert may be inserted between the genes. For 3′,3′ overlaps the synthetic insert may comprise the overlapping region.

For 5′, 3′ overlaps (i.e. pairs of genes in the same orientation, comprising an upstream gene and a downstream gene) a synthetic insert may be inserted between the genes. For 5′,3′ overlaps the synthetic insert may comprise: (i) a stop codon; (ii) about 200 bp, or 20-100 bp, or 20-50 bp, from upstream of the overlapping region; and (iii) the overlapping region. Preferably, the synthetic insert comprises: (i) a stop codon; (ii) about 20 bp from upstream of the overlapping region; and (iii) the overlapping region. This preserves the sequence of the RBS for the downstream ORF and the distance between this RBS and its start codon.

In preferred embodiments the stop codon is in frame with the original start site for the downstream gene. Preferably the stop codon is TAA.

A development of REXER, CONEXER (Zurcher et al., 2023; WO2023/078997), uses conjugative transfer to iterate the steps of REXER and facilitate creation of larger episomes comprising recoded DNA. CONEXER also placed the excision sites of the insert in the backbone of the vector, such that the integrated DNA always includes one or more nucleotides derived from backbone sequence. This permits excision of the insert from the episomal replicon using the same pair of CRISPR spacers-‘universal spacers’-which can be used to perform REXER at any target locus with a given episomal replicon backbone. This massively simplifies the introduction of synthetic DNA into a target nucleic acid, such as an E. coli genome, and enables accelerated methods for large-scale genome engineering and whole genome synthesis. The arrangement of spacers that bind within the backbone and minimize the distance of the cleavage site from the junction between the backbone and the insert leads to the excision of an insert flanked by 6 base pairs of the backbone on each end. These 6 bp sequences are not commonly homologous to the region of the target nucleic acid flanking the regions complementary to the insert. The presence of short non-homologous ends on the excised synthetic DNA does not impede recombination and integration efficiency, and scarless integration is still achieved.

Our copending application P41907 describes methods not dependent on CRISPR-Cas methodologies.

DNA sequences or polynucleotides according to the invention can be used to replace two or more corresponding sections of an E. coli genome in a parent strain. The number of replaced section can be two or more, 3 or more, 4 or more, 5 or more, 6 or more, 7 or more, 8 or more and so on up to 38 or 39 or 40 sections. Advantageously, substantially all of the E. coli genome is replaced with recoded DNA sequence according to the invention.

It will be understood that replacement of codons requires assignment of codons to coding sequences in genetic maps of the E. coli in question, and that such assignment of codons may be inaccurate and may change with improvements in analysis of E. coli genomes. Accordingly, “substantially all” refers to all known codons at the filing date hereof and may not be definitive; further codons may be discovered, and/or codons presently assigned may be designated as non-coding.

Transfer RNA (tRNA) cognate for the removed leucine codon(s) may be removed from the E. coli cell, for example by deleting the gene encoding it. Thus, advantageously, the gene encoding tRNA Leu GAG (LeuU) is deleted, and the genes encoding LeuP and LeuW are either deleted or engineered such that their decoding specificity excludes CTT and CTC.

Non-canonical amino acids may also be referred to as non-proteinogenic amino acids or unnatural amino acids (UAA) are not particularly limited; they can be chosen from any non-canonical amino acid for which a suitable orthogonal tRNA/aaRS pair is available. Suitable non-proteinogenic amino acid and UAAs will be well known to those of skill in the art, for example those disclosed in Neumann, H., 2012. FEBS letters, 586 (15), pp. 2057-2064; and Liu, C. C. and Schultz, P. G., 2010. Annual review of biochemistry, 79, pp. 413-444. In some embodiments the non-proteinogenic amino acid and/or UAAs are selected from one or more of: p-Acetylphenylalanine, m-Acetylphenylalanine, O-allyltyrosine, Phenylselenocysteine, p-Propargyloxyphenylalanine, p-Azidophenylalanine, p-Boronophenylalanine, O-methyltyrosine, p-Aminophenylalanine, p-Cyanophenylalanine, m-Cyanophenylalanine, p-Fluorophenylalanine, p-lodophenylalanine, p-Bromophenylalanine, p-Nitrophenylalanine, L-DOPA, 3-Aminotyrosine, 3-lodotyrosine, p-Isopropylphenylalanine, 3-(2-Naphthyl) alanine, Biphenylalanine, Homoglutamine, D-tyrosine, p-Hydroxyphenyllactic acid, 2-Aminocaprylic acid, Bipyridylalanine, HQ-alanine, p-Benzoylphenylalanine, o-Nitrobenzylcysteine, o-Nitrobenzylserine, 4,5-Dimethoxy-2-nitrobenzylserine, o-Nitrobenzyllysine, 0-Nitrobenzyltyrosine, 2-Nitrophenylalanine, Dansylalanine, p-Carboxymethylphenylalanine, 3-Nitrotyrosine, Sulfotyrosine, Acetyllysine, Methylhistidine, 2-Aminononanoic acid, 2-Aminodecanoic acid, Pyrrolysine, Cbz-lysine, Boc-lysine and Allyloxycarbonyllysine.

Uses of the E. coli Cells of the Invention

The E. coli cells of the invention may be used to synthesise polymers or proteins that include a first and/or second non-canonical amino acid. When associated with other mutations, such as stop codon reassignment or serine codon reassignment as described in WO2020/229592, up to 5 non-canonical amino acids may be included. In particular embodiments, the prokaryotic cells of the invention may be used to synthesise polymers or proteins that include two, three, four or five different types of non-canonical amino acid. The non-canonical amino acids may be any as disclosed herein or known elsewhere in the art.

Thus, in an aspect of the invention, there is provided a method of synthesising a polymer, comprising:
i) providing an E. coli cell of the invention, wherein the E. coli cell comprises a first orthogonal aminoacyl-tRNA synthetase-tRNA pair; and contacting said E. coli cell with a nucleic acid sequence encoding a polymer, said nucleic acid sequence comprising the first type of sense codon;
ii) incubating the E. coli cell in the presence of a first non-canonical amino acid, wherein the first non-canonical amino acid is a substrate for the first orthogonal aminoacyl-tRNA synthetase; and
iii) incubating the E. coli cell to allow incorporation of the first non-canonical amino acid into the polymer via the first orthogonal aminoacyl-tRNA synthetase-tRNA pair.

In a particular embodiment, there is provided a method of synthesising a polymer, comprising:
i) providing an E. coli cell of the invention, wherein the E. coli cell comprises first and second orthogonal aminoacyl-tRNA synthetase-tRNA pairs; and contacting said E. coli cell with a nucleic acid sequence encoding a polymer, said nucleic acid sequence comprising the first type of sense codon and/or the second type of sense codon;
ii) incubating the E. coli cell in the presence of a first and a second non-canonical amino acid, wherein the first non-canonical amino acid is a substrate for the first orthogonal aminoacyl-tRNA synthetase and the second non-canonical amino acid is a substrate for the second orthogonal aminoacyl-tRNA synthetase; and
iii) incubating the E. coli cell to allow incorporation of the first non-canonical amino acid into the polymer via the first orthogonal aminoacyl-tRNA synthetase-tRNA pair and the incorporation of the second non-canonical amino acid into the polymer via the second first orthogonal aminoacyl-tRNA synthetase-tRNA pair.

In an embodiment, there is provided a method of synthesising a polymer, comprising: i) providing an E. coli cell of the invention, wherein the prokaryotic cell comprises first, second, and third orthogonal aminoacyl-tRNA synthetase-tRNA pairs; and contacting said E. coli cell with a nucleic acid sequence encoding a polymer, said nucleic acid sequence comprising a first type of sense codon, a second type of sense codon, and/or a first type of stop codon;
ii) incubating the E. coli cell in the presence of a first, a second, and a third non-canonical amino acid, wherein the first non-canonical amino acid is a substrate for the first orthogonal aminoacyl-tRNA synthetase, the second non-canonical amino acid is a substrate for the second orthogonal aminoacyl-tRNA synthetase, and the third non-canonical amino acid is a substrate for the third orthogonal aminoacyl-tRNA synthetase;
iii) incubating the E. coli cell to allow incorporation of the first non-canonical amino acid into the polymer via the first orthogonal aminoacyl-tRNA synthetase-tRNA pair, incorporation of the second non-canonical amino acid into the polymer via the second first orthogonal aminoacyl-tRNA synthetase-tRNA pair, and/or incorporation of the third non-canonical amino acid into the polymer via the third orthogonal aminoacyl-tRNA synthetase-tRNA pair.

In an embodiment, there is provided a method of synthesising a polymer, comprising: i) providing an E. coli cell of the invention, wherein the prokaryotic cell comprises first, second, third and fourth orthogonal aminoacyl-tRNA synthetase-tRNA pairs; and contacting said E. coli cell with a nucleic acid sequence encoding a polymer, said nucleic acid sequence comprising a first type of sense codon, a second type of sense codon, a third type of sense codon and/or a first type of stop codon;
ii) incubating the E. coli cell in the presence of a first, a second, a third and a fourth non-canonical amino acid, wherein the first non-canonical amino acid is a substrate for the first orthogonal aminoacyl-tRNA synthetase, the second non-canonical amino acid is a substrate for the second orthogonal aminoacyl-tRNA synthetase, the third non-canonical amino acid is a substrate for the third orthogonal aminoacyl-tRNA synthetase and the fourth non-canonical amino acid is a substrate for the fourth orthogonal aminoacyl-tRNA synthetase;
iii) incubating the E. coli cell to allow incorporation of the first non-canonical amino acid into the polymer via the first orthogonal aminoacyl-tRNA synthetase-tRNA pair, incorporation of the second non-canonical amino acid into the polymer via the second first orthogonal aminoacyl-tRNA synthetase-tRNA pair, incorporation of the third non-canonical amino acid into the polymer via the third first orthogonal aminoacyl-tRNA synthetase-tRNA pair, and/or incorporation of the fourth non-canonical amino acid into the polymer via the fourth orthogonal aminoacyl-tRNA synthetase-tRNA pair.

In an embodiment, there is provided a method of synthesising a polymer, comprising: i) providing an E. coli cell of the invention, wherein the prokaryotic cell comprises first, second, third, fourth and fifth orthogonal aminoacyl-tRNA synthetase-tRNA pairs; and contacting said E. coli cell with a nucleic acid sequence encoding a polymer, said nucleic acid sequence comprising a first type of sense codon, a second type of sense codon, a third type of sense codon, a fourth type of sense codon and/or a first type of stop codon;
ii) incubating the E. coli cell in the presence of a first, a second, a third, a fourth and a fifth non-canonical amino acid, wherein the first non-canonical amino acid is a substrate for the first orthogonal aminoacyl-tRNA synthetase, the second non-canonical amino acid is a substrate for the second orthogonal aminoacyl-tRNA synthetase, the third non-canonical amino acid is a substrate for the third orthogonal aminoacyl-tRNA synthetase, the fourth non-canonical amino acid is a substrate for the fourth orthogonal aminoacyl-tRNA synthetase and the fifth non-canonical amino acid is a substrate for the fifth orthogonal aminoacyl-tRNA synthetase;
iii) incubating the E. coli cell to allow incorporation of the first non-canonical amino acid into the polymer via the first orthogonal aminoacyl-tRNA synthetase-tRNA pair, incorporation of the second non-canonical amino acid into the polymer via the second first orthogonal aminoacyl-tRNA synthetase-tRNA pair, incorporation of the third non-canonical amino acid into the polymer via the third first orthogonal aminoacyl-tRNA synthetase-tRNA pair, incorporation of the fourth non-canonical amino acid into the polymer via the fourth orthogonal aminoacyl-tRNA synthetase-tRNA pair, and/or incorporation of the fifth non-canonical amino acid into the polymer via the fifth orthogonal aminoacyl-tRNA synthetase-tRNA pair.

The contacting of the E. coli cell with the nucleic acid sequence may take any form that would provide the nucleic acid to the prokaryotic cell such that the nucleic acid sequence may be decoded to produce a polymer, such as a protein.

The E. coli cells for use in the present method may be any as described herein, wherein the prokaryotic cells comprise first and, optionally, second, third fourth and/or fitfth orthogonal aminoacyl-tRNA synthetase-tRNA pairs. For instance, in an embodiment, the E. coli cell for use in the present method is an E. coli cell that does not express a first endogenous tRNA, a second endogenous tRNA, optionally des not express a third and/or fourth endogenous tRNA, and a first endogenous release factor; and wherein a first and a second, and optionally a third and/or a fourth, type of sense codon has been recoded within the genome of the E. coli cell such that the endogenous tRNA(s) are dispensable, and a first type of stop codon has been recoded within the genome of the E. coli cell such that the first endogenous release factor is dispensable. The nature of the recoding may be as described for any E. coli cell of the invention disclosed herein. The first orthogonal tRNA is capable of decoding the first type of sense codon, the second orthogonal tRNA is capable of decoding the second type of sense codon, and the third orthogonal tRNA is capable of decoding the first type of stop codon.

In a particular embodiment, the prokaryotic cell for use in the present method is an E. coli cell that does not express tRNASerUGA, RNASer CGA, or RF-1, occurrences of the sense codon TCA have been recoded such that tRNASerUGA is dispensable (e.g. occurrences of TCA in essential genes of the parent strain have been replaced with AGT), occurrences of the sense codon TCG have been recoded such that tRNASerCGA is dispensable (e.g. occurrences of TCG in essential genes of the parent strain have been replaced with AGC), and occurrences of the stop codon TAG have been recoded such that RF-1 is dispensable (e.g. occurrences of TAG in essential genes of the parent strain have been replaced with TAA). The bacterium has been modified to include first, second, and third orthogonal aminoacyl-tRNA synthetase-tRNA pairs capable of decoding the TCA, TCG, and TAG codons and capable of incorporating a first, second, and third non-canonical amino acid into such sites during polymer formation.

In particular embodiments, the parental prokaryotic cell for use in the present method is Syn6143, Syn6143 (ev3), Syn6143 (ev4), or Syn6143 (ev5). See WO2022/248061.

Prokaryotic cells, e.g. E. coli, are not typically able to incorporate most eukaryotic post-translational modifications, such as ubiquitination, glycosylation and phosphorylation, nor are they typically capable of other eukaryotic maturation processes, and proteolytic protein maturation. In addition, correct disulphide bond formation and lipolysaccharide contaminations can be troublesome (see Ovaa, H., 2014. Frontiers in chemistry, 2, p. 15). However, therapeutic proteins, such as antibodies, enzymes and cytokines commonly carry post-translational modifications and disulphide bonds, and often require proteolytic maturation to attain their correctly folded state. Thus, the majority of therapeutic proteins are produced in eukaryotic and mammalian cell systems. However, expression in prokaryotic cells e.g. E. coli is in general cheaper, more susceptible to genetic modifications, and versatile with regard to mutant library development, and suitable for industrial scale fermentation (Ovaa, H., 2014. Frontiers in chemistry, 2, p. 15).

Thus, in some embodiments the synthesised polymers are therapeutic polypeptides, optionally wherein mammalian protein modifications have been introduced via one or more non-canonical amino acids. The synthesised polymer may be a protein, such as a therapeutic protein, comprising at least three, four or five distinct types of non-canonical amino acid.

In some embodiments the synthesised polymers, polypeptides, or proteins may include at least one non-canonical amino acid directly adjacent to another non-canonical amino acid. This polymer, polypeptide, or protein may include a chain of two, three, four, five, six, seven, eight, nine, ten, 15, 20, or more non-canonical amino acids directly adjacent to each other. The chain may comprise two, three, four or five distinct non-canonical amino acids.

In some embodiments, a polypeptide may be excisable from the synthesised polymer. For instance, the nucleic acid may encode cleavage or cleavable sites either side of the intended product. The excised product may be entirely made of non-canonical amino acids or may comprise one or more canonical amino acid. The excised product may be the chain of non-canonical amino acids described herein.

The synthesised polymer, including the linear or cyclic polymers, or protein may be purified after the method of synthesis. The purification may take any form known in the art in order to separate the polymer or protein from the cells and culture in which it was made.

The E. coli cells of the invention can be used to generate products that are not obtainable by any other methods. As such, in an aspect of the invention, there is provided a polymer obtained from or obtainable by the methods of the invention. In an embodiment, the polymer comprises at least one first non-canonical amino acid, at least one second non-canonical amino acid, and optionally at least one third, fourth or fifth non-canonical amino acid, wherein the first, second, third, fourth and fifth non-canonical amino acids are different from each other. The polymer may comprise least one non-canonical amino acid directly adjacent to another non-canonical amino acid. The polymer may include a chain of two, three, four, five, six, seven, eight, nine, ten, 15, 20, or more non-canonical amino acids directly adjacent to each other. The polymer may be a macrocycle or may be linear. Macrocycles of the present invention may comprise four, five, six, seven, eight, nine, ten or more monomers.

The prokaryotic cells of the invention may be suitable for the production of a target polymer or protein incorporating at least one non-canonical amino acid, wherein the rate of production is at least 5%, 10%, 15%, 25%, 35%, 50%, 60%, 70%, 80%, 90%, 100%, 110%, or 140% of the rate of production of the wild type control.

The prokaryotic cells of the invention may be suitable for the production of a target polymer or protein incorporating at least one non-canonical amino acid, wherein yield is at least 0.5, 1, 2, 5, 7.5, 10, 15, 20, or 25 mg/l.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O′D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press.

### EXAMPLES

### Example 1

### Recoding the Cell Division Operon

We identified the E. coli MDS42 cell division operon (from 89,062 to 106,507) as a test region for leucine recoding. The 17 kbp region (from 89,062 to 106,507) of interest contains fifteen genes, five of which overlap in distinct reading frames. 12 of the 15 genes are essential, and the genes include genes expressed at different levels, genes encoding membrane proteins, several proteins with differing expression ratios, and a high number of Leucine codons. To allow independent recoding of each gene, while leaving potential cis-regulatory regions within the overlap intact, we adopted a new architecture in which 200 nucleotides upstream of the start codon of each overlapping ORF are duplicated (see WO2018/020248).

We designed a DNA sequence in which targeted leucine codons are replaced with various synonymous codon alternatives and implemented these recoding schemes within all the fifteen genes in this new architecture.

The DNA for each scheme was synthesized de novo. Short synthetic DNA fragments (~1 kb) were assembled into a pSC101 plasmid using GoldenGate assembly and Gibson assembly. The assembled synthetic DNA (~10 kb) were used as precursor fragments for assembly into a bacterial artificial chromosome (BAC) using recombination in S. cerevisiae, where synthetic insert is coupled to a sacB-cat double selection cassette, and the BAC backbone contains an additional rpsL negative marker, as in WO2018/020248. The resulting BAC was introduced into an E. coli strain, MDS42rpsLK43R comprising a genomic phes *-hygR double selection cassette immediately upstream of the cell division operon, introduced through lambda red mediated recombination. The REXER2 protocol, as described in WO2018/020248, was employed to test genomic recoding by replacement of the targeted 17 kbp cell division operon region with the leucine codon-recoded DNA sequence.

After REXER of each recoding scheme, colonies were picked and successful recombination was assessed by genotyping of the selection marker swap at the relevant loci by colony PCR, as described in WO2018/020248, as well as selective growth assays of the resulting clones in different selective media.

Clones that had selection marker genotypes and phenotypes consistent with REXER-mediated recombination across the entire ~17 kbp region of interest were carried forward. Genomic DNA was extracted from these clones using Biosearch Technologies QuickExtract DNA Extraction Solution. DNA libraries for next-generation sequencing were prepared using the Illumina Nextera XT kit, and sequenced in an Illumina NextSeq2000, as per manufacturer's instructions. The presence of recoded alleles across the ~17 kbp region was analyzed, with the aim of identifying clones that had undergone complete recoding across the region.

The results are shown in Table 1 below:

| | | | | Recoding | |
|---|---|---|---|---|---|
| | | | | effi- | % |
| | | Codon | | ciency | Fully |
| Amino | Recoding | substitution | | (num, of | recoded |
| acid | scheme | From | To | clones) | clones |
| Leucine | CDO_Leu_5 | CTG | CTC | 0/24 | 0% |
| | | CTA | CTT | | |
| | CDO_Leu_8 | CTG | CTC | 0/24 | 0% |
| | | CTA | CTC | | |
| | CDO_Leu_9 | CTT | TTG | 18/24 | 75% |
| | | CTC | TTG | | |
| | CDO_Leu_12 | CTT | TTA | 22/24 | 92% |
| | | CTC | TTG | | |

These results demonstrate that recoding schemes targeting the CTT and CTC leucine codons (CDO_Leu_9 and CDO_Leu_12) were significantly more successful than those targeting CTG and CTA codons (CDO_Leu_5 and CDO_Leu_8). The most successful recoding scheme was CDO_Leu_12, which achieved a 92% success rate in producing fully recoded clones.

For CDO_Leu_9 and CDO_Leu_12, we set out to assess the impact of recoding in the fitness of the post-REXER cells, by measuring their growth curves compared to a the non-recoded MDS42 parent. Post-REXER clones were streaked from glycerol stocks onto selective agar plates and 3 colonies of each were inoculated separately into 2×YT media supplemented with 5 μg/μL tetracycline and 20 μg/μL chloramphenicol, and grown overnight in 96-well plates. The next day, these were diluted 1 in 40 and the growth curves were analysed in an Agilent BioTek LogPhase 600 microplate reader, at 37° C. with shaking at 800 rpm for a duration of 24 hours with OD₆₀₀ measurement intervals every 10 minutes

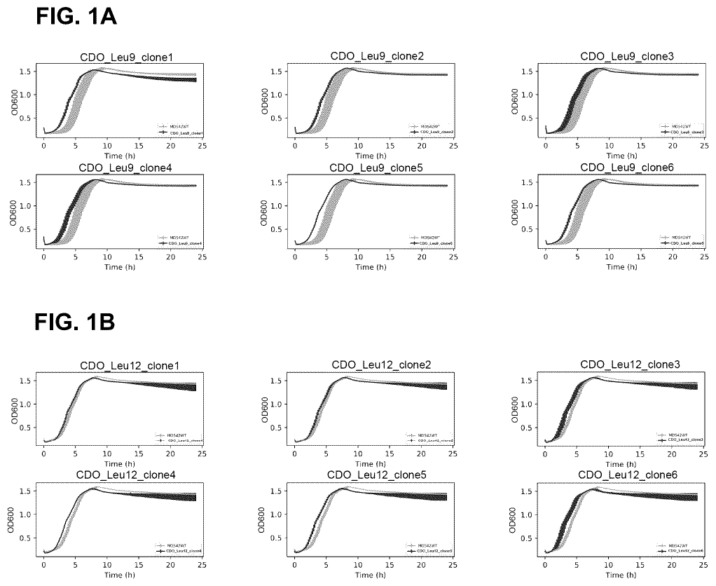

FIGS. 1A and 1B show the growth curves of fully recoded clones for CDO_Leu_9 and CDO_Leu_12, compared to the non-recoded parent stain MDS42. 6 representative clones are shown for each recoding schemes. The data shows that the impact of implementing these recoding schemes across the essential cell division operon of E. coli is minimal, highlighting the suitability of these schemes for synonymous codon compression.

These experiments demonstrate the feasibility of recoding large sections of the E. coli genome, including essential genes, by replacing specific leucine codons with synonymous alternatives. The success of the CDO_Leu_9 and CDO_Leu_12 recoding schemes provides a foundation for further genome-wide recoding efforts and potential reassignment of the removed leucine codons to non-canonical amino acids.

1. A DNA sequence encoding one or more essential genes of an E. coli organism, said DNA sequence comprising at least 17 Kb and being recoded such that a leucine codon is changed to an alternative codon, wherein said leucine codon is CTT or CTC.

2. The DNA sequence according to claim 1, wherein the alternative codon is a synonymous codon.

3. The DNA sequence according to claim 1, wherein the codon CTT is changed to TTG or TTA.

4. The DNA sequence according to claim 1, wherein the codon CTC is changed to TTG.

5. The DNA sequence according to claim 1, wherein said DNA sequence encodes at least 10 essential genes.

6. An engineered E. coli organism comprising a DNA sequence encoding one or more essential genes of the E. coli organism, said DNA sequence comprising at least 17 Kb and being recoded such that a leucine codon is changed to an alternative codon, wherein said leucine codon is CTT or CTC, and wherein said E. coli is viable.

7. The engineered E. coli organism according to claim 6, wherein two or more sections of the endogenous genome have each been replaced with a DNA sequence encoding one or more essential genes of the E. coli organism, each of said DNA sequences comprising at least 17 Kb and being recoded such that a leucine codon is changed to an alternative codon, wherein said leucine codon is CTT or CTC.

8. The engineered E. coli organism according to claim 6, wherein the codon CTT is changed to TTG or TTA.

9. The engineered E. coli organism according to claim 6, wherein the codon CTC is changed to TTG.

10. The engineered E. coli organism according to claim 6, wherein said DNA sequence encodes at least 10 essential genes.

11. The engineered E. coli organism according to claim 6, wherein all instances of at least one stop codon are removed and the cognate release factor is removed.

12. The engineered E. coli organism according to claim 6, wherein all instances of one or more serine codons are removed and the cognate tRNA genes are not present in the recoded genome.

13. An engineered E. coli organism comprising a recoded genome wherein a leucine codon at all instances within a template genome is changed to an alternative codon and said leucine codon is reassigned to a non-canonical amino acid, wherein said leucine codon is CTT or CTC, and wherein said E. coli is viable.

14. The engineered E. coli organism according to claim 13, wherein the cognate tRNA to said leucine codon which is changed to an alternative codon is not present in the recoded genome.

15. The engineered E. coli organism according to claim 13, wherein the codon CTT is changed to TTG or TTA.

16. The engineered E. coli organism according to claim 13, wherein the codon CTC is changed to TTG.

17. The engineered E. coli organism according to claim 13, wherein both leucine codons CTT and CTC are removed and wherein the LeuU tRNA gene is not present in the recoded genome.

18. The engineered E. coli organism according to claim 13, wherein all instances of at least one stop codon are removed and the cognate release factor is removed.

19. The engineered E. coli organism according to claim 13, wherein all instances of one or more serine codons are removed and the cognate tRNA genes are not present in the recoded genome.

20. A method for producing a protein comprising one or more non-canonical amino acids, said method comprising expressing a nucleic acid encoding said protein in the engineered E. coli organism of claim 6.

21. The method according to claim 20, wherein the protein comprises one, two, three, four or five non-canonical amino acids, wherein said non-canonical amino acids are different.

22. A method for producing a protein comprising one or more non-canonical amino acids, said method comprising expressing a nucleic acid encoding said protein in the engineered E. coli organism of claim 13.

23. The method according to claim 22, wherein the protein comprises one, two, three, four or five non-canonical amino acids, wherein said non-canonical amino acids are different.
